(19) United States
(12) Patent Application Publication
(10) Pub. No.: US 2022/0056008 A1
(43) Pub. Date: Feb. 24, 2022

(54) PROCESS AND INTERMEDIATES FOR THE SYNTHESIS OF VOXELOTOR

(71) Applicant: CRYSTAL PHARMA, S.A.U., Boecillo - Valladolid (ES)

(72) Inventors: Juan José FERREIRO GIL, Boecillo - Valladolid (ES); Jesús Miguel IGLESIAS RETUERTO, Boecillo - Valladolid (ES); Antonio LORENTE BONDE-LARSEN, Boecillo - Valladolid (ES)

(21) Appl. No.: 17/416,991

(22) PCT Filed: Dec. 20, 2019
(86) PCT No.: PCT/EP2019/086635

§ 371 Date: Jun. 21, 2021

(30) Foreign Application Priority Data
Dec. 21, 2018 (EP) 18382987.8

(51) Int. Cl. C07D 401/04

(52) U.S. Cl. CPC C07D 401/04

(57) ABSTRACT

The invention relates to a process for the preparation of Voxelotor, or a salt or solvate thereof, according to the following scheme (Formula 1).

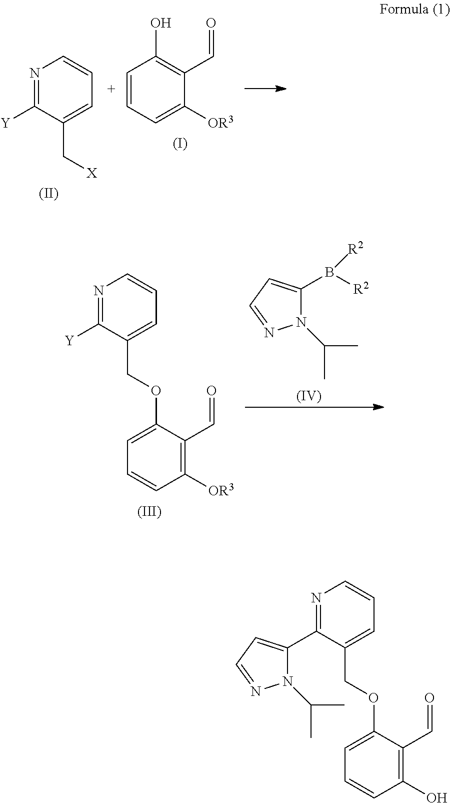

### FIELD OF THE INVENTION

The invention relates to a process for the preparation of Voxelotor and derivatives thereof and to intermediates useful in the synthesis of these compounds.

### BACKGROUND OF THE INVENTION

Voxelotor and pharmaceutical compositions comprising it are suitable as allosteric modulators of hemoglobin, for their use in treating disorders mediated by hemoglobin and disorders that would benefit from tissue and/or cellular oxygenation.

Sickle cell disease is a group of disorders that affects hemoglobin, the molecule in red blood cells that delivers oxygen to cells throughout the body. People with this disorder have atypical hemoglobin molecules called hemoglobin S, which can distort red blood cells into a sickle, or crescent, shape. When red blood cells sickle, they break down prematurely, which can lead to anemia. Anemia can cause shortness of breath, fatigue, and delayed growth and development in children.

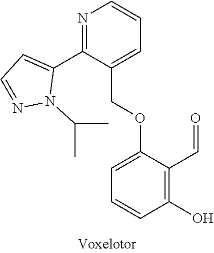 [SMILES: CC(C)n1nccc1-c1ncccc1COc1cccc(O)c1C=O]

Several synthetic processes for preparing Voxelotor and intermediates thereof have been disclosed.

Preparation of Voxelotor was first disclosed in WO 2013/102,142. The process disclosed therein requires several synthetic steps for preparing the pyrazole ring and further chromatographic separation of the resulting isomers. Voxelotor is finally obtained through alkylation of the chloride derivative with 2,6-dihydroxy-benzaldehyde.

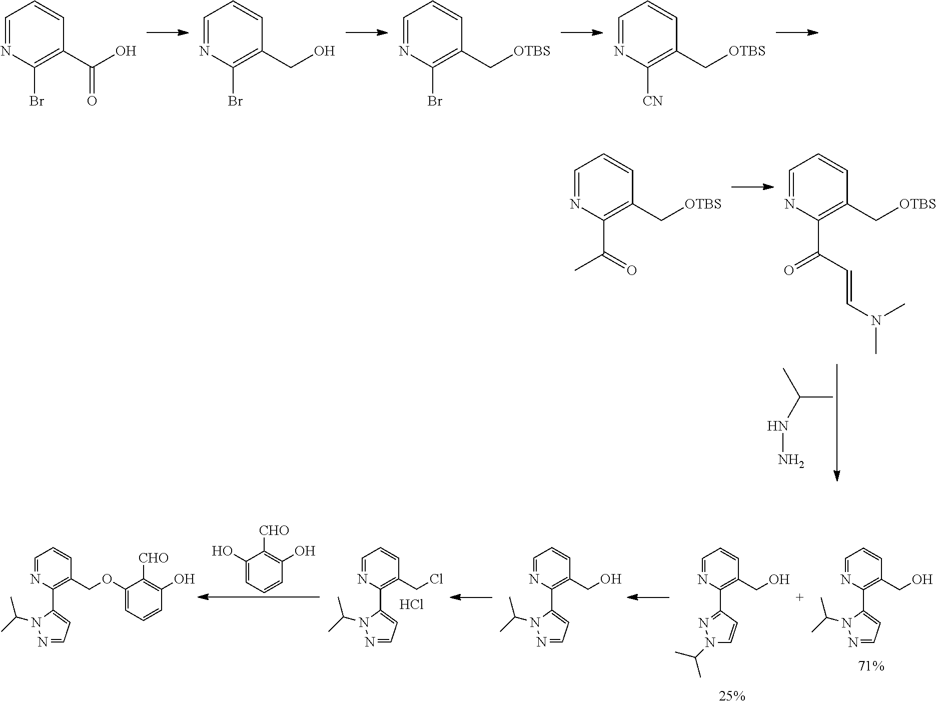

WO 2014/150276 discloses a more straightforward process for preparing the intermediate in the synthesis of Voxelotor (INT-4) comprising a Suzuki cross-coupling reaction.

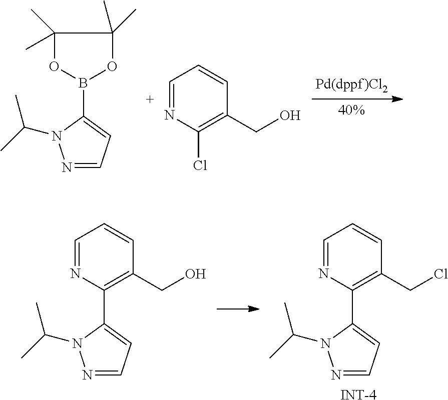

Documents WO 2015/031285 and ACS Medicinal Chemistry Letters 2017, 8(3), 321-326 disclose the use of mono-protected 2,6-dihydroxy-benzaldehyde in order to avoid bis-alkylation side products.

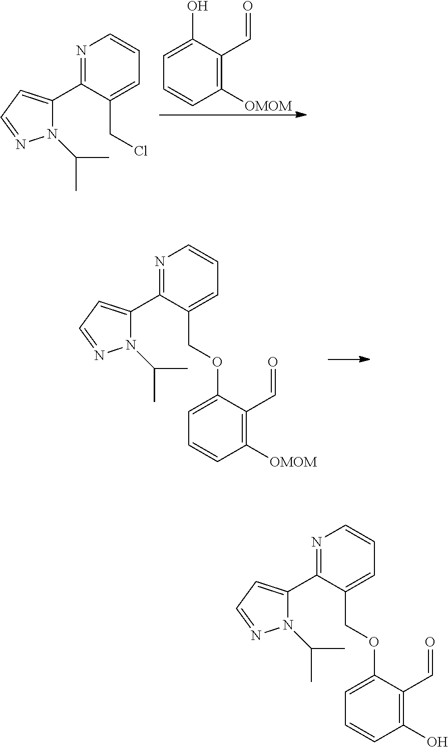

The mono-protected compound can be obtained through a multi-step sequence from resorcinol or from bromo-resorcinol.

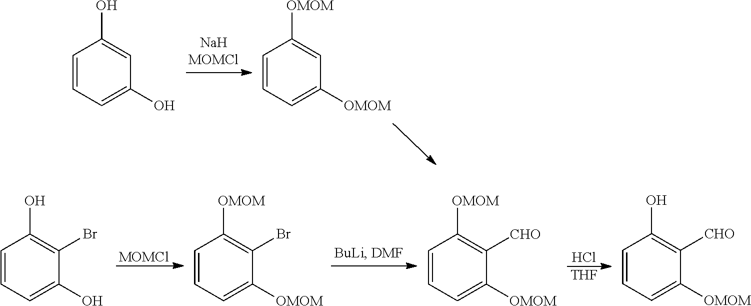

In these processes MOMCl, which is carcinogen, is used to prepare the MOM-protected compounds.

These documents also describe introduction of the phenyl ether through Mitsunobu reaction.

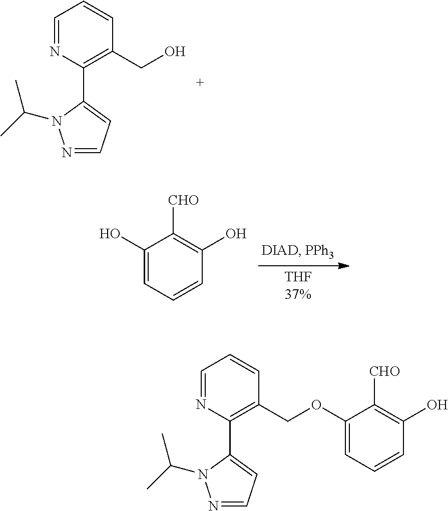 [SMILES: CC(C)n1nccc1-c1ncccc1CO ; O=Cc1c(O)cccc1O ; CC(C)n1nccc1-c1ncccc1COc1cccc(O)c1C=O]

Though several processes for the preparation of Voxelotor and intermediates thereof have been disclosed, they require many synthetic steps and/or give rise to the desired product in low yield.

It is therefore necessary to develop a new process for obtaining Voxelotor as well as key intermediates in its synthesis which overcome all or part of the problems associated with the known processes belonging to the state of the art.

### SUMMARY OF THE INVENTION

The invention faces the problem of providing a new process for the preparation of Voxelotor and intermediates thereof. In particular, the inventors have found a very efficient process for the synthesis of Voxelotor which comprises first reacting the compound of formula (I) with a compound of formula (II) and then introducing the pyrazole ring via a Suzuki coupling reaction of the resulting compound with a boron compound of formula (IV).

In contrast to the processes from the prior art, in the process of the invention the expensive boron compound of formula (IV) is used at a later stage of the synthesis and so can be used in a lower amount than in the prior art.

Additionally, the process of the invention provides a more efficient synthesis of Voxelotor, leading to the desired in compound in very high yield and purity, even without the need of purification by column chromatography.

Consequently, the process of the present invention for the synthesis of Voxelotor is more convenient and suitable for its industrial application.

Accordingly, in a first aspect the invention is directed to a process for preparing Voxelotor, or a salt or solvate thereof, comprising:
(a) reacting a compound of formula (I)

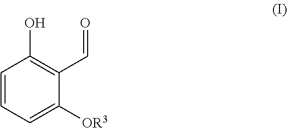

or a salt or solvate thereof, wherein R³ represents hydrogen or a hydroxyl protecting group,
with a compound of formula (II)

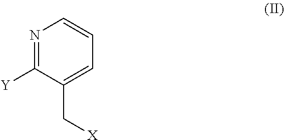

or a salt or solvate thereof, wherein
X is selected from OH, Cl, Br, I, OTf, OTs and OMs, and
Y is selected from Cl, Br, I, OTf and OMs;
to obtain a compound of formula (III)

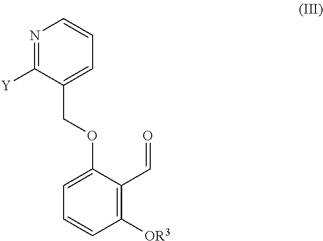

or a salt or solvate thereof;
(b) reacting a compound of formula (III), or a salt or solvate thereof, with a compound of formula (IV)

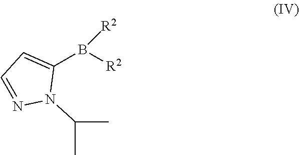

or a salt or solvate thereof, wherein each R² is independently selected from the group consisting of OH, C₁₋₆ alkyl, C₃₋₇ cycloalkyl, C₁₋₆ alkoxyl, or together they form a C₂₋₃ alkylenedioxy group optionally substituted by C₁₋₆ alkyl, or a benzyldioxy group optionally substituted by C₁₋₆ alkyl, or the —B(R²)₂ group is —BF₃K,
to provide a compound of formula (V)

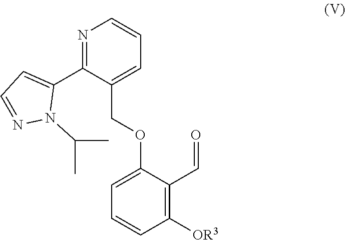

or a salt or solvate thereof; and
(c) If R³ in the compound of formula (V) or a salt or solvate thereof is a hydroxyl protecting group, cleaving the hydroxyl protecting group to provide Voxelotor or a salt or solvate thereof.

In another aspect the invention is directed to a compound of formula (III′)

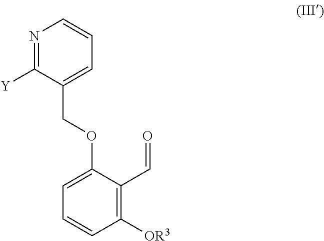

or a salt or solvate thereof, wherein
Y is selected from I, OTf and OMs, and
R³ represents hydrogen or a hydroxyl protecting group.

### DETAILED DESCRIPTION OF THE INVENTION

The term “C₁-C₆ alkyl” refers to a linear or branched alkane derivative containing from 1 to 6, preferably from 1 to 3 (“C₁-C₃ alkyl”), carbon atoms and which is bound to the rest of the molecule through a single bond. Illustrative examples of alkyl groups include methyl, ethyl, n-propyl, i-propyl, n-butyl, i-butyl, t-butyl, pentyl, hexyl. Preferably, it is methyl or ethyl.

The term “C₃-C₇ cycloalkyl” refers to a radical derived from cycloalkane containing from 3 to 7, preferably from 3 to 6 (“C₃-C₆ cycloalkyl”) carbon atoms. Illustrative examples of cycloalkyl groups include cyclopropyl, cyclobutyl, cyclopentyl, cyclohexyl, etc.

The term “C₁-C₆ alkoxyl” designates an alkyl group as defined above having between 1 and 6 carbon atoms, more preferably between 1 and 3 carbon atoms (“C₁-C₃ alkoxyl”), linked to the rest of the molecule through oxygen. Examples of alkoxy include methoxy, ethoxy, isopropoxy, tertbutoxy, and the like.

The term “C₂-C₃ alkylenedioxy” designates a divalent group represent by —O—R—O—, where R is an alkylene group having two or three carbon atoms. These carbon atoms can be optionally substituted with one or more C₁-C₆ alkyl groups. Examples of C₂-C₃ alkylenedioxy groups include —O—CH₂—CH₂—O—, —O—CH(CH₃)—CH(CH₃)—O—, —O—C(CH₃)₂—CH(CH₃)—O—, —O—C(CH₃)₂—C(CH₃)₂—O—, —O—CH₂—CH₂—CH₂—O—, —O—CH₂—C(CH₃)₂—CH₂—O— and —O—C(CH₃)₂—CH₂—C(CH₃)₂—O—.

The term “C₆-C₁₀ aryl” refers to an aromatic group having between 6 and 10, preferably 6 or 10 carbon atoms, comprising 1 or 2 aromatic nuclei fused to one another. Illustrative examples of aryl groups include phenyl, naphthyl, indenyl, phenanthryl, etc. Preferably, it is phenyl

The term “halogen” refers to bromine, chlorine, iodine or fluorine.

The term “heterocyclyl” refers to a saturated or partially unsaturated monocyclic or bicyclic system containing from 3 to 10, preferably 5 to 7, ring atoms containing one or more, specifically one, two, three or four ring heteroatoms independently selected from N, O, and S, and the remaining ring atoms being carbon.

The term “heteroaryl” refers to an aromatic monocyclic or bicyclic system containing from 3 to 10, preferably 5 to 7, ring atoms containing one or more, specifically one, two, three or four ring heteroatoms independently selected from O, N and S, and the remaining ring atoms being carbon.

The term “hydroxyl protecting group” (HPG) refers to a group blocking the OH function for subsequent reactions that can be removed under controlled conditions. Hydroxyl protecting groups are well known in the art. Illustrative examples of hydroxyl protecting groups have been described by Green T W et al. in “Protective Groups in Organic Synthesis”, 3rd Edition (1999), Ed. John Wiley & Sons. Virtually any hydroxyl protecting group can be used to put the invention into practice. Illustrative, non-limiting examples of HPGs include:
silyl ethers [—Si(R)(R′)(R″)]. R, R′ and R″ can be independently selected from C₁-C₆ alkyl, C₃-C₇ cycloalkyl, C₆-C₁₀ aryl, C₁-C₆ alkoxy and halogen. Examples of silyl ethers include trimethylsilyl ether, triethylsilyl ether, tert-butyldimethylsilyl ether, tert-butyldiphenylsilyl ether, tri-isopropylsilyl ether, diethylisopropylsilyl ether, hexyldimethylsilyl ether, triphenylsilyl ether, di-tert-butylmethylsilyl ether;
ethers [—R], including alkoxy and aryloxy methyl ethers [—CH₂—OR]. R can be selected from C₁-C₆ alkyl, C₆-C₁₀ aryl and (C₆-C₁₀)aryl(C₁-C₆)alkyl. Examples of ethers include methyl ether, tert-butyl ether, benzyl ether, p-methoxybenzyl ether, 3,4-dimethoxybenzyl ether, trityl ether, allyl ether, methoxymethyl ether, 2-methoxyethoxymethyl ether, benzyloxymethyl ether, p-methoxybenzyloxymethyl ether, 2-(trimethylsilyl)ethoxymethyl ether; tetrahydropyranyl and related ethers;
esters [—COR]. R can be selected from C₁-C₆ alkyl, C₆-C₁₀ aryl and (C₆-C₁₀)aryl(C₁-C₆)alkyl. Examples of esters include acetate ester, benzoate ester, pivalate ester, methoxyacetate ester, chloroacetate ester, levulinate ester; and
carbonates [—COOR]. R can be selected from C₁-C₆ alkyl, C₆-C₁₀ aryl and (C₆-C₁₀)aryl(C₁-C₆)alkyl. Examples of carbonates include benzyl carbonate, p-nitrobenzyl carbonate, tert-butyl carbonate, 2,2,2-trichloroethyl carbonate, 2-(trimethylsilyl)ethyl carbonate, allyl carbonate.

As understood in this technical area, there may be a certain degree of substitution in the aforementioned radicals. Therefore, there may be substitution in any of the groups of the present invention. The previous groups can be substituted in one or more available positions with one or more substituents. Said substituents include, for example and in non-limiting sense, C₁₋₆ alkyl, C₃₋₇ cycloalkyl, C₆-C₁₀ aryl, 3- to 10-membered heterocyclyl, 3- to 10-membered heteroaryl, halogen, —CN, NO₂, CF₃, —N(Rₐ)(R_{b}), —OR_{c}, —SR_{d}, —C(O)Rₑ, —C(O)OR_{f}, —C(O)N(R_{g})(Rₕ), —OC(O)Rᵢ; wherein Rₐ, R_{b}, R_{c}, R_{d}, Rₑ, R_{f}, R_{g}, Rₕ and Rᵢ are independently selected from hydrogen, C₁-C₆ alkyl, C₆-C₁₀ aryl, 3- to 10-membered heterocyclyl, 3- to 10-membered heteroaryl and trifluoromethyl.

The invention also provides “salts” of the compounds described herein. By way of illustration, said salts can be acid addition salts, base addition salts or metal salts, and can be synthesized from the parent compounds containing a basic or acid moiety by means of conventional chemical processes known by the persons skilled in the art. Such salts are generally prepared, for example, by reacting the free acid or base forms of said compounds with a stoichiometric amount of the suitable base or acid in water or in an organic solvent or in a mixture of the two. Non-aqueous media such as ether, ethyl acetate, ethanol, acetone, isopropanol or acetonitrile are generally preferred. Illustrative examples of acid addition salts include inorganic acid addition salts such as, for example, hydrochloride, hydrobromide, hydroiodide, sulfate, nitrate, phosphate, etc., organic acid addition salts such as, for example, acetate, maleate, fumarate, citrate, oxalate, succinate, tartrate, malate, mandelate, methanesulfonate, p-toluenesulfonate, camphorsulfonate, etc. Illustrative examples of base addition salts include inorganic base salts such as, for example, ammonium salts and organic base salts such as, for example, ethylenediamine, ethanolamine, N,N-dialkylethanolamine, triethanolamine, glutamine, amino acid basic salts, etc. Illustrative examples of metal salts include, for example, sodium, potassium, calcium, magnesium, aluminium and lithium salts.

The term “solvate” according to this invention is to be understood as meaning any form of the compound which has another molecule (most likely a polar solvent) attached to it via non-covalent bonding. Examples of solvate include hydrates and alcoholates, e.g. methanolates. Solvation methods are generally known in the state of the art.

The term “organic solvent” includes for example cyclic and acyclic ethers (e.g. Et₂O, iPr₂O, tBu₂O, MeOtBu, 1,4-dioxane, 1,3-dioxolane, 1,2-dimethoxyethane (DME), tetrahydrofuran (THF), methyltetrahydrofuran), hydrocarbon solvents (e.g. pentane, hexane, heptane), halogenated solvents (e.g. dichloromethane, dichloroethane, chloroform), aromatic solvents (e.g. toluene, xylene), esters (e.g. EtOAc, BuOAc), ketones (e.g. acetone, methylethyl ketone, cyclohexanone), nitriles (e.g. acetonitrile), amides (e.g. DMF, DMA, NMP), alcohols (e.g. methanol, ethanol, propanol, i-propanol, t-butanol), sulfoxides (DMSO) and mixtures thereof.

In an aspect, the invention is directed to a process for preparing Voxelotor, or a salt or solvate thereof, which comprises:
(a) reacting a compound of formula (I)

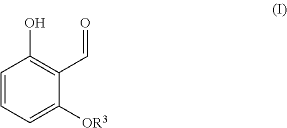

or a salt or solvate thereof, wherein R³ represents hydrogen or a hydroxyl protecting group,
with a compound of formula (II)

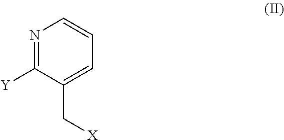

or a salt or solvate thereof, wherein
X is selected from OH, Cl, Br, I, OTf, OTs and OMs, and
Y is selected from Cl, Br, I, OTf and OMs;
to obtain a compound of formula (III)

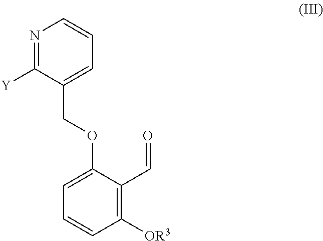

or a salt or solvate thereof;
(b) reacting a compound of formula (III), or a salt or solvate thereof, with a compound of formula (IV)

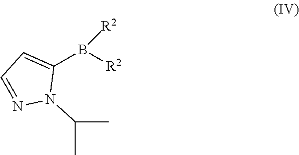

or a salt or solvate thereof, wherein each R² is independently selected from the group consisting of OH, C₁₋₆ alkyl, C₃₋₇cycloalkyl, C₁₋₆ alkoxyl, or together they form a C₂₋₃ alkylenedioxy group optionally substituted by C₁₋₆ alkyl, or a benzyldioxy group optionally substituted by C₁₋₆ alkyl, or the —B(R²)₂ group is —BF₃K,
to provide a compound of formula (V)

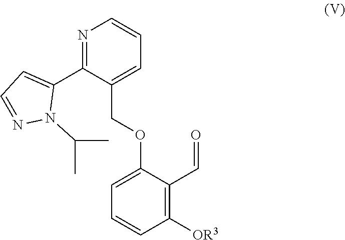

or a salt or solvate thereof; and
(c) If R³ in the compound of formula (V) or a salt or solvate thereof is a hydroxyl protecting group, cleaving the hydroxyl protecting group to provide Voxelotor or a salt or solvate thereof.

In an embodiment, R³ is a hydroxyl protecting group, such as an ether, a silyl ether, an ester or a carbonate.

In an embodiment, X is selected from Cl and OH.

In another embodiment, Y is Cl.

In a preferred embodiment, Y is Cl and X is selected from Cl and OH; or Y is Cl and X is Cl. More preferably, X is Cl and Y is Cl.

In a preferred embodiment of the invention, each R² in the compound of formula (VIII) is independently selected from the group consisting of OH, C₁₋₆ alkoxyl, or together they form a C₂₋₃ alkylenedioxy group optionally substituted by C₁₋₆ alkyl. More preferably, each R² is OH.

In a particular embodiment, R³ in the compound of formula (I) is a group of formula —CH₂—O—R¹, wherein R¹ is a C₁₋₆ alkyl group. Preferably, R¹ is Me or Et. More preferably, R¹ is Me.

In an embodiment, the compound of formula (I) wherein R³ is a group of formula —CH₂—O—R¹, or a salt or solvate thereof, is obtained by a process comprising:
(a) reacting 1,3-benzenediol

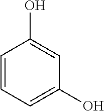

with a compound of formula R¹—O—CH₂-halide, wherein R¹ is a C₁₋₆ alkyl group, generated in situ by reacting a compound of formula R¹—O—CH₂—O—R¹ with a halide source; to obtain a compound of formula (VI)

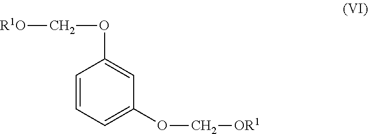

(b) formylating a compound of formula (VI), to obtain a compound of formula (VII)

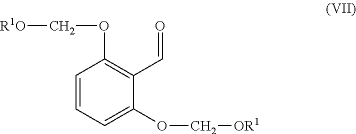

and
(c) cleaving one alkoxymethyl ether group in the compound of formula (VII), to obtain a compound of formula (I), or a salt or solvate thereof, wherein R³ is a group of formula —CH₂—O—R¹

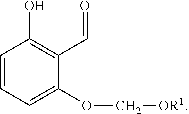

Particular and preferred embodiments for the above-mentioned reactions are as disclosed below.

Reaction of a Compound of Formula (I) with a Compound of Formula (II)

The compound of formula (V), or a salt or solvate thereof, is obtained by reacting a compound of formula (I), or a salt or solvate thereof, with a compound of formula (II), or a salt or solvate thereof.

This reaction can be carried out under alkylation reaction conditions or under Mitsunobu reaction conditions. Preferably, it is carried out under alkylation reaction conditions.

(i) Alkylation Reaction

In a preferred embodiment, X in the compound of formula (II), or a salt or solvate thereof, is selected from Cl, Br, I, OTf, OTs and OMS and the reaction with the compound of formula (I), or a salt or solvate thereof, is performed under alkylation reaction conditions.

Preferably, the reaction is carried out in the presence of a base and an organic solvent. Suitable bases include, for example, alkaline and alkaline earth metal carbonates, bicarbonates, phosphates, C₁₋₆ alkoxides, hydroxides and hydrides; preferably alkaline carbonates and hydrides, such as Na₂CO₃, K₂CO₃, Cs₂CO₃ or NaH. Suitable organic solvents include, for example, DMF, DMSO, NMP, acetonitrile, acetone, methylethyl ketone, THF, CH₂Cl₂, EtOAc, BuOAc.

In an embodiment, the reaction is carried out in the presence of an inorganic base, such as for example alkaline and alkaline earth metal carbonates, bicarbonates, phosphates, C₁₋₆ alkoxides, hydroxides and hydrides; preferably alkaline carbonates and hydrides, such as Na₂CO₃, K₂CO₃, Cs₂CO₃ or NaH.

In a particular embodiment, the reaction is carried out in the presence of an inorganic base and an organic solvent selected from an ether (e.g. Et₂O, iPr₂O, tBu₂O, MeOtBu, 1,4-dioxane, 1,3-dioxolane, 1,2-dimethoxyethane (DME), tetrahydrofuran (THF), methyltetrahydrofuran), a halogenated solvent (e.g. dichloromethane, dichloroethane, chloroform), an ester (e.g. EtOAc, BuOAc), a ketone (e.g. acetone, methylethyl ketone, cyclohexanone), a nitrile (e.g. acetonitrile), an amide (e.g. DMF, DMA, NMP), a sulfoxide (DMSO) and mixtures thereof.

In a particular embodiment, the reaction is carried out in the presence of an inorganic base, such as Na₂CO₃, K₂CO₃, Cs₂CO₃ or NaH, and DMF.

The base is typically used in an amount ranging from 1.0 and 8.0 equivalents for each equivalent of compound of formula (V), preferably from 1.5 to 5.0 equivalents.

In an embodiment, the reaction is performed at a temperature between 0° C. and 150° C., preferably between 30° C. and 120° C., more preferably between 40° C. and 90° C.

Mitsunobu Reaction

In another embodiment, X in the compound of formula (II), or a salt or solvate thereof, is OH and the reaction with the compound of formula (I), or a salt or solvate thereof, is performed under Mitsunobu reaction conditions.

In an embodiment, the reaction is performed in the presence of a first reagent selected from the group consisting of triphenylphosphine, tributylphosphine and trimethylphosphine, and a second reagent selected from the group consisting of group consisting of diisopropyl azodicarboxylate (DIAD), di-tert-butyl azodicarboxylate (DBAD), diethyl azodicarboxylate (DEAD), di-p-chlorobenzyl azodicarboxylate (DCAD), 1,1′-(azodicarbonyl)dipiperidine (ADDP), N,N,N′,N′-tetraisopropylazodicarboxamide (TIPA), N, N, N′,N′-tetramethylazodicarboxamide (TMAD) and 4,7-dimethyl-3,4,5,6,7,8-hexahydro-1,2,4,7-tetrazocin-3,8-dione (DHTD). Preferably, in the presence of triphenylphosphine and DIAD or DEAD.

Preferably, the reaction is performed in an organic solvent, such as THF or toluene.

It can be carried out, for example, at a temperature between −30° C. and 70° C., preferably, between 0 and 50° C.

Reaction of a Compound of Formula (III) with a Compound of Formula (IV)—Suzuki Reaction

Preferably, the reaction is carried out in the presence of a base and a palladium catalyst.

Suitable bases include, for example, alkaline and alkaline earth metal carbonates, bicarbonates, phosphates, acetates, alkoxides, hydroxides and halides; preferably alkaline carbonates, bicarbonates and phosphates, such as Na₂CO₃, K₂CO₃, Cs₂CO₃, NaHCO₃, Na₃PO₄ or K₃PO₄.

In a preferred embodiment, the base is an inorganic base, such as alkaline or alkaline earth metal carbonate, bicarbonate or phosphate; preferably alkaline carbonates, bicarbonates and phosphates, such as Na₂CO₃, K₂CO₃, Cs₂CO₃, NaHCO₃, Na₃PO₄ or K₃PO₄, which can be used in any of their forms, including grounded into powder form. More preferably the base is NaHCO₃ or Na₂CO₃, even more preferably the base is NaHCO₃.

The base is typically used in an amount ranging from 1.0 and 8.0 equivalents for each equivalent of compound of formula (III), preferably from 1.5 to 5.0 equivalents.

Suitable palladium catalysts include, Pd(0) catalysts and Pd(II) catalysts that are reduced in situ to Pd(0). In an embodiment, the palladium catalyst is selected from Pd(PPh₃)₄, Pd₂(dba)₃, Pd(OAc)₂, Pd(P^{t}Bu₃)₂, Pd(PCy₃)₂, Pd(PPh₃)₂Cl₂, Pd(P(o-tol)₃)₂Cl₂, Pd(PCy₃)₂Cl₂, Pd(P^{t}Bu₂Ph)₂Cl₂, Pd(P^{t}BuCy₂)₂Cl₂, Pd(P^{t}Bu₂ⁿBu)₂Cl₂, Pd(amphos)Cl₂ (amphos=di-tert-butyl(4-dimethylaminophenyl)phosphine), Pd(dppe)₂Cl₂ (dppe=(1,2-bis(diphenylphosphino)ethane), Pd(dppp)₂Cl₂ (dppp=(1,2-bis(diphenylphosphino)propane), Pd(dppb)₂Cl₂ (dppb=(1,2-bis(diphenylphosphino)butane), Pd(dppf)Cl₂ (dppf=1,1′-bis(diphenylphosphino)ferrocene), Pd(dtbpf)Cl₂ (dtbpf=1,1′-bis(di-tert-butylphosphino)ferrocene), Pd(dcypp)Cl₂ (dcypp=bis(dicyclohexylphosphino)propane), [PdBr(P^{t}Bu₃)]₂, Pd/C with PPh₃, Pd(PhCN)₂Cl₂, Pd(CH₃CN)₂Cl₂, and solvates thereof.

In a preferred embodiment, the palladium catalyst is selected from Pd(PPh₃)₂Cl₂, Pd(amphos)Cl₂, Pd(PCy₃)₂Cl₂ and Pd(PCy₃)₂. More preferably, it is selected from Pd(PPh₃)₂Cl₂ and Pd(amphos)Cl₂. Even more preferably, it is Pd(amphos)Cl₂.

Typically, the amount of the Pd catalyst is from 0.01% mol to 20% mol, such as from 0.1% mol to 10% mol.

The inventors have found that the Suzuki reaction can be carried out using very low amounts of the Pd catalyst, especially for preferred Pd catalysts defined above. In an embodiment, the Pd catalyst is used in an amount between 0.01 to 15 wt % based on the weight of the compound of formula (III). In an embodiment, it is used in an amount from 0.1 to 10 wt %, or from 0.1 to 5 wt %, based on the weight of the compound of formula (III).

Further, in a particular embodiment the reaction proceeds in the presence of water, an organic solvent, or mixtures thereof.

According to a particular embodiment, this reaction is carried out in the presence of an organic solvent or mixture of solvents, for example, an ether (e.g., THF, 2-methyltetrahydrofuran, DME, dioxane, 1,3-dioxolane), a nitrile (e.g. acetonitrile), an alcohol (e.g. methanol, ethanol, propanol, i-propanol, t-butanol), an aromatic solvent (e.g., toluene, xylene) or mixtures thereof and, optionally, in the presence of water.

In a preferred embodiment, the reaction is carried out in the presence of water and an ether (e.g., THF, 2-methyltetrahydrofuran, DME, dioxane, 1,3-dioxolane), a nitrile (e.g. acetonitrile) or an alcohol (e.g. methanol, ethanol, propanol, i-propanol, t-butanol). More preferably, in the presence of water and dioxane or in the presence of water and acetonitrile or in the presence of water and i-propanol. In an embodiment, the ratio of organic solvent to water ranges from 20:1 to 1:5, preferably from 10:1 to 1:1.

In a particular embodiment, the reaction is carried out using NaHCO₃ or Na₂CO₃ as the base, preferably NaHCO₃, and in the presence of an organic solvent and water.

In a particular embodiment, the reaction is carried out using NaHCO₃ or Na₂CO₃ as the base, preferably NaHCO₃, and in the presence of water and an ether (e.g., THF, 2-methyltetrahydrofuran, DME, dioxane, 1,3-dioxolane), a nitrile (e.g. acetonitrile) or an alcohol (e.g. methanol, ethanol, propanol, i-propanol, t-butanol).

In a particular embodiment, the reaction is carried out using NaHCO₃ or Na₂CO₃ as the base, preferably NaHCO₃, a Pd catalyst selected from Pd(PPh₃)₂Cl₂, Pd(amphos)Cl₂, Pd(PCy₃)₂Cl₂ and Pd(PCy₃)₂, and in the presence of an organic solvent and water.

In a particular embodiment, the reaction is carried out using NaHCO₃ or Na₂CO₃ as the base, preferably NaHCO₃, a Pd catalyst selected from Pd(PPh₃)₂Cl₂, Pd(amphos)Cl₂, Pd(PCy₃)₂Cl₂ and Pd(PCy₃)₂, and in the presence of water and an ether (e.g., THF, 2-methyltetrahydrofuran, DME, dioxane, 1,3-dioxolane), a nitrile (e.g. acetonitrile) or an alcohol (e.g. methanol, ethanol, propanol, i-propanol, t-butanol).

In a preferred embodiment, the reaction is carried out using NaHCO₃ or Na₂CO₃ as the base, preferably NaHCO₃, a Pd catalyst selected from Pd(PPh₃)₂Cl₂, Pd(amphos)Cl₂, Pd(PCy₃)₂Cl₂ and Pd(PCy₃)₂, and in the presence of water and an ether (e.g., THF, 2-methyltetrahydrofuran, DME, dioxane, 1,3-dioxolane), preferably water and dioxane.

In a further embodiment, the reaction is carried out in the presence of NaHCO₃, Pd(PPh₃)₂Cl₂ and a mixture of water and dioxane.

In a further embodiment, the reaction is carried out in the presence of NaHCO₃, Pd(PPh₃)₂Cl₂ and a mixture of water and acetonitrile.

In a further embodiment, the reaction is carried out in the presence of NaHCO₃, Pd(amphos)Cl₂ and a mixture of water and THF.

In a further embodiment, the reaction is carried out in the presence of NaHCO₃, Pd(amphos)Cl₂ and a mixture of water and dioxane.

In a further embodiment, the reaction is carried out in the presence of Na₂CO₃, Pd(PCy₃)₂ and a mixture of water and i-propanol.

In an embodiment, the reaction is carried out in the presence of NaHCO₃, Pd(PPh₃)₂Cl₂, water and an ether, preferably dioxane.

The reaction can be carried out under heating, for example at a temperature comprised between 40° C. and 130° C., preferably between 60° C. and 110° C.

The compound of formula (IV) is typically used in an amount ranging from 1.0 and 3.0 equivalents for each equivalent of compound of formula (III), preferably from 1.0 to 2.0 equivalents.

In a particular embodiment, each R² in the compound of formula (IV) is independently selected from the group consisting of OH, C₁₋₆ alkoxyl, or together they form a C₂₋₃ alkylenedioxy group optionally substituted by C₁₋₆ alkyl. Preferably, each R² in the compound of formula (VIII) is OH, methoxy, ethoxy, i-propoxy or, together, form an ethylendioxy, tetramethylethylenedioxy, propylenedioxy, dimethylpropylendioxy, trimethylpropylendioxy or tetramethylpropylendioxy group. In an embodiment, each R² is OH.

In a preferred embodiment, the R² groups in the compound of formula (IV) form together a C₂₋₃ alkylenedioxy group optionally substituted by C₁₋₆ alkyl, such as an ethylendioxy, tetramethylethylenedioxy, propylenedioxy, dimethylpropylendioxy, trimethylpropylendioxy or tetramethylpropylendioxy group. Preferably, they form a tetramethylethylenedioxy group.

In a preferred embodiment, the Pd catalyst is Pd(amphos)Cl₂ and the R² groups in the compound of formula (IV) form together a C₂₋₃ alkylenedioxy group, preferably a tetramethylethylenedioxy group.

In a further preferred embodiment, the reaction is carried out in the presence of NaHCO₃, Pd(amphos)Cl₂, a mixture of water and an ether (preferably THF or dioxane) and a compound of formula (IV) wherein the R² groups form together a C₂₋₃ alkylenedioxy group, preferably a tetramethylethylenedioxy group.

In a preferred embodiment, Y in the compound of formula (III) is Cl.

### Cleavage of the Hydroxyl Protecting Group

Conversion of the compound of formula (V) wherein R³ is a hydroxyl protecting group into Voxelotor can be performed as disclosed in the prior art (e.g. WO 2015/031285, ACS Medicinal Chemistry Letters 2017, 8(3), 321-326).

Additionally, deprotection of the hydroxyl groups in the compounds of the invention can be performed by conventional methods known by those skilled in the art (e.g. Green T W et al. in “Protective Groups in Organic Synthesis”, 3rd Edition (1999), Ed. John Wiley & Sons (ISBN 0-471-16019-9)).

For example, compounds wherein OR³ represents an ester (R³═COR) or a carbonate (R³═COOR) can be easily deprotected by hydrolysis in basic or acid media according to well-established procedures of the state of the art.

Compounds wherein OR³ represents a silyl ether (R³═Si(R)(R′)(R″) can be deprotected by the use of fluoride reagents such as fluoride salts or HF, acid media, oxidizing media, etc.

Compounds wherein OR³ represents an ether (R³═R, CH₂OR) can be easily deprotected through hydrolysis in acid media (for example, for methyl ethers (R³═CH₂OR)), hydrogenation (for example, for benzyl ethers), oxidation (for example, for aryl ethers), etc.

In a particular embodiment, OR³ is a C₁₋₆ alkoxymethyl ether (R³═CH₂O(C₁₋₆ alkyl)). Preferably, this hydroxyl protecting group is cleaved by acid hydrolysis, for example by treatment with an acid such as HCl, H₂SO₄, HBr, HF, HNO₃, acetic acid, trifluoroacetic acid, methanesulfonic acid, trifluoromethanesulfonic acid, p-toluenesulfonic acid.

This reaction can be carried out in the presence of an organic solvent, water or mixtures thereof.

In an embodiment, this reaction can be carried out at a temperature between −20° C. and 120° C. Preferably, between 0° C. and 100° C.

Conversion of 1,3-Benzenediol into a Compound of Formula (VI)

In an embodiment, compound of formula (VI) is obtained by reacting 1,3-benzenediol (resorcinol) with a compound of formula R¹—O—CH₂-halide, wherein R¹ is a C₁₋₆ alkyl group, generated in situ by reacting a compound of formula R¹—O—CH₂—O—R¹ with a halide source.

In this way, since the compound R¹—O—CH₂-halide (e.g. MOM-Cl) is generated in situ, its direct manipulation is avoided. This is advantageous over prior art methods where MOM-Cl, which is carcinogenic, is directly used as hydroxyl protecting agent.

In an embodiment, the reaction of a compound of formula R¹—O—CH₂—O—R¹ wherein R¹ is a C₁₋₆ alkyl group with a halide source is carried out in the presence of a Lewis acid and optionally an organic solvent

Suitable halide sources include acyl halides, (COCl)₂ and SOCl₂. In an embodiment, the halide source is selected from (C₁₋₆ alkyl)COCl, (C₆₋₁₀ aryl)COCl, (C₁₋₆ alkyl)COBr, (C₆₋₁₀ aryl)COBr, (COCl)₂ and SOCl₂. In a particular embodiment, the halide source is selected from AcCl, AcBr, (COCl)₂ and SOCl₂. In an embodiment, it is AcCl.

Preferably, the halide source is a chloride or bromide source; more preferably a chloride source.

In an embodiment, the halide source is used in an amount from 1.0 to 3.0 equivalents based on the compound of formula R¹—O—CH₂—O—R¹; preferably from 1.0 to 2.0 equivalents.

Suitable Lewis acids include, for example, ZnBr₂, Zn(OTf)₂, ZnI₂, ZnCl₂ and Zn(OAc)₂. In an embodiment, the Lewis acid is ZnBr₂.

In an embodiment, the Lewis acid is used in an amount from 0.0001 to 20.0 wt % based on the compound of formula R¹—O—CH₂—O—R¹; preferably from 0.01 to 10.0 wt %.

In an embodiment, the reaction is carried out neat (i.e. in the absence of an inert solvent). In another embodiment, the reaction is carried out in the presence of an organic solvent, such as an ether (e.g. Et₂O, iPr₂O, tBu₂O, MeOtBu, 1,4-dioxane, 1,3-dioxolane, DME, THF, methyltetrahydrofuran), a hydrocarbon solvent (e.g. pentane, hexane, heptane), a halogenated solvent (e.g. dichloromethane, dichloroethane, chloroform), an aromatic solvent (e.g. toluene, xylene), an ester (e.g. EtOAc, BuOAc), a nitrile (e.g. acetonitrile), an amide (e.g. DMF, DMA, NMP), a sulfoxide (DMSO) and mixtures thereof.

This reaction for in situ generation of the compound R¹—O—CH₂-halide can be carried out at a temperature between 0° C. and 60° C., preferably between 10° C. and 40° C.

In an embodiment, the reaction is carried out in the presence of AcCl and ZnBr₂.

Preferably, R¹ is Me and the halide source is a chloride source, so that the in situ generated compound is MOM-Cl.

In a particular embodiment, compound of formula (VI) is obtained by reacting 1,3-benzenediol (resorcinol) with the in situ generated compound of formula R¹—O—CH₂-halide, in the presence of a base and an organic solvent.

Suitable bases include organic bases (such as pyridine, trimethylamine, triethylamine, diisopropylethylamine, N-methyl-2-pyrrolidone) and inorganic bases (such as alkaline and alkaline earth metal carbonates, bicarbonates, phosphates and hydrides; preferably alkaline metal carbonates and hydrides, such as Na₂CO₃, K₂CO₃, Cs₂CO₃ or NaH).

The reaction can be carried out in the presence of an organic solvent such as an ether (e.g. Et₂O, iPr₂O, tBu₂O, MeOtBu, 1,4-dioxane, 1,3-dioxolane, DME, THF, methyltetrahydrofuran), a halogenated solvent (e.g. dichloromethane, dichloroethane, chloroform), an ester (e.g. EtOAc, BuOAc), a ketone (e.g. acetone, methylethyl ketone, cyclohexanone), a nitrile (e.g. acetonitrile), an amide (e.g. DMF, DMA, NMP), a sulfoxide (DMSO) and mixtures thereof.

In an embodiment, the base is used in an amount from 2 to 10 equivalents based on the 1,3-benzenediol; preferably, from 2 to 6 equivalents.

In an embodiment, the compound of formula R¹—O—CH₂-halide is used in an amount from 2 to 10 equivalents based on the 1,3-benzenediol; preferably, from 2 to 6 equivalents.

The reaction can be carried out at a temperature between −20° C. and 100° C.; preferably from 0° C. to 60° C.

### Formylation of a Compound of Formula (VI)

Formylation of a compound of formula (VI) to obtain a compound of formula (VII) can be carried out as disclosed in the prior art, for example in WO 2013/102,142, WO 2014/150,276, WO 2015/031,285 and ACS Medicinal Chemistry Letters 2017, 8(3), 321-326.

In an embodiment, compound of formula (VII) is obtained by reacting a compound of formula (VI) with a formylating agent, such as N,N-diallylformamide, formic acid, a formic acid ester (e.g. methyl formate, ethyl formate), formylmorpholine, formylpiperidine or formylpiperazine.

In an preferred embodiment, the formylating agent is a N,N-dialkylformamide, such as N,N-dimethylformamide or N,N-diethylformamide; preferably, it is DMF.

In an embodiment, the formylation reaction is carried out in the presence of a lithium base, such as MeLi, nBuLi, sBuLi, tBuLi or LDA.

In a preferred embodiment, the formylation reaction is carried out in the presence of a lithium base and a N,N-dialkylformamide, preferably a lithium base and DMF.

The reaction can be carried out in the presence of an organic solvent, preferably an ether (e.g. Et₂O, iPr₂O, tBu₂O, MeOtBu, 1,4-dioxane, 1,3-dioxolane, 1,2-dimethoxyethane (DME), tetrahydrofuran (THF), methyltetrahydrofuran), more preferably THF.

The reaction can be carried out a temperature between −78° C. and 50° C., preferably between −78° C. and 30° C.

### Cleavage of One Alkoxymethyl Ether Group in the Compound of Formula (VII)

A compound of formula (I), or a salt or solvate thereof, wherein R³ is a group of formula —CH₂—O—R¹

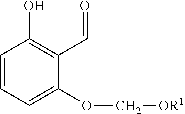

wherein R¹ is a C₁₋₆ alkyl group,

can be obtained from a compound of formula (VII) by cleavage of one alkoxymethyl ether group.

This reaction can be carried out as disclosed in the prior art, for example WO 2013/102,142, WO 2014/150,276, WO 2015/031285 and ACS Medicinal Chemistry Letters 2017, 8(3), 321-326.

In a particular embodiment, the alkoxymethyl ether group is cleaved by acid hydrolysis, for example by treatment with an acid such as HCl, H₂SO₄, HBr, HF, HNO₃, acetic acid, trifluoroacetic acid, methanesulfonic acid, trifluoromethanesulfonic acid, p-toluenesulfonic acid; preferably HCl.

In an embodiment, the acid is used in an amount between 1.0 and 1.5 equivalents, preferably between 1.0 and 1.3, equivalents based on the compound of formula (VII).

This reaction can be carried out in the presence of an organic solvent, water or mixtures thereof. Preferably, the organic solvent is an ether (e.g. Et₂O, iPr₂O, tBu₂O, MeOtBu, 1,4-dioxane, 1,3-dioxolane, 1,2-dimethoxyethane (DME), tetrahydrofuran (THF), methyltetrahydrofuran), more preferably THF.

In an embodiment, this reaction can be carried out at a temperature between −20° C. and 120° C.; preferably, between 0° C. and 100° C.; more preferably, between 0° C. and 50° C.

If needed during the processes of the invention, protection and/or deprotection reactions of the hydroxyl groups can be performed at any stage of the synthesis. The most suitable stage for said protection and/or deprotection can be readily determined by those skilled in the art.

### Compounds of Formula (III)

Compounds of formula (III) are useful intermediates for the preparation of Voxelotor.

Therefore, in another aspect, the invention is directed to a compound of formula (III′)

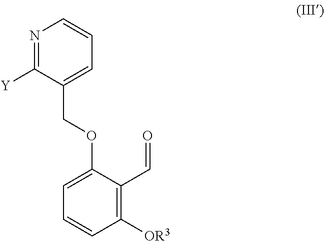

or a salt or solvate thereof, wherein
Y is selected from I, OTf and OMs, and
R³ represents hydrogen or a hydroxyl protecting group.

In a preferred embodiment R³ is a group of formula R or CH₂—OR, wherein R is selected from C₁-C₆ alkyl, C₆-C₁₀ aryl and (C₆-C₁₀)aryl(C₁-C₆)alkyl. Examples of OR³ groups include methyl ether, tert-butyl ether, benzyl ether, p-methoxybenzyl ether, 3,4-dimethoxybenzyl ether, trityl ether, allyl ether, methoxymethyl ether, 2-methoxyethoxymethyl ether, benzyloxymethyl ether, p-methoxybenzyloxymethyl ether, 2-(trimethylsilyl)ethoxymethyl ether; tetrahydropyranyl and related ethers. In a particular embodiment, R³ is a methoxymethyl group (MOM).

### EXAMPLES

### Preparation of Compound (I)

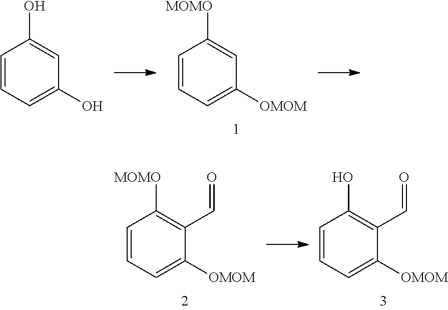

### Preparation of Compound 1

To a 25 mL flask at 10° C. containing dimethoxymethane (96.6 mL) and ZnBr₂ (0.116 g) was added slowly (0.5 h) acetyl chloride (38.9 mL). The mixture was stirred at room temperature over 2 h, then a mixture of resorcinol (15.0 g), DMF (225 mL) and K₂CO₃ (75.4 g) was added slowly at room temperature. The mixture obtained was heated at 60/65° C. and stirred until reaction was finished. The mixture was cooled to room temperature and the solid obtained was filtered off. Water (160 mL) was added to the liquid phase. The solvent was removed on a rotavap at 40° C. under vacuum. The aqueous layer was extracted with isopropyl ether (75 mL three times). The combined organic layers were concentrated to afford a solid that was dissolved with isopropyl ether (75 mL) and was washed with brine (30 mL twice). The organic layer was concentrated to afford 12.2 g of a solid of compound 1.

### Preparation of Compound 2

A solution of THF (92.5 mL) and compound 1 (18.5 g) was stirred at −10° C. Then, hexyl lithium (52.7 mL, 2.3 M) was added slowly and stirred for 30 minutes. DMF (9.4 mL) was added slowly. Water (37 mL) was added. The mixture was then stirred for 1 h at room temperature. The mixture was extracted with methylene chloride (55 mL). The organic layer was washed with an aqueous solution of sodium chloride 25%. The organic layer was concentrated to afford a solid of compound 2 (23.7 g).

### Preparation of Compound 3

To a solution of compound 2 (7.4 g) in THF (52.0 mL) was added slowly conc. HCl (3.3 mL, 12 N). The solution was stirred at rt until the reaction was complete. The mixture was added to an aqueous solution of NaCl 25% (37 mL). The mixture was extracted with methylene chloride (37 mL twice). The organic phase was washed with an aqueous solution of NaCl 25% and an aqueous solution of NaHCO₃ 7% (17 mL). The organic layer was concentrated to afford a solid of compound 3 (4.65 g).

### Synthesis of Voxelotor

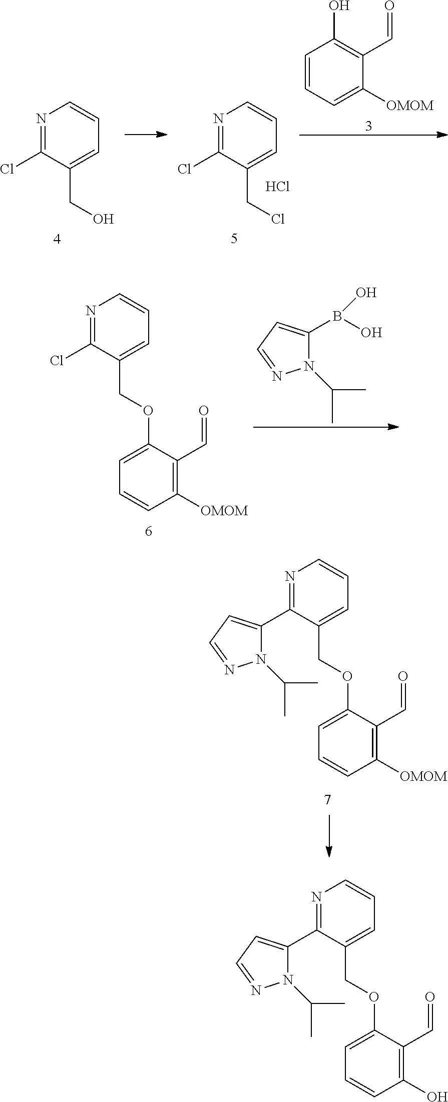

### Preparation of Compound 5

SOCl₂ (8.13 mL) was added at rt to (2-chloropyridin-3-yl)methanol 4 (8 g) in DCM (80 mL). The reaction mixture was stirred at rt until the end of the reaction and concentrated to dryness. The crude solid was suspended in toluene and concentrated to dryness. The process was repeated three times and dried under vacuum to give an oil, 2-chloro-3-(chloromethyl)pyridine hydrochloride 5 (11.25 g), which was used in the next step without further purification.

### Preparation of Compound 6

A mixture of compound 5 (1.5 g, 1.03 equiv), compound 3 (1.59 g), and K₂CO₃ (4.39 g, 4 equiv) in DMF (18 mL) was heated at 60/65° C. and was stirred until reaction was finished. The mixture was cooled and added to water (100 mL) dropwise. The precipitate was filtered, washed with water and dried under high vacuum to give compound 6 (2.12 g, 86%) as a solid.

1HNMR (400 MHz, CDCl₃) δ 8.57 (d, 1H), 10.61 (s, 1H), 8.33 (t, 2H), 7.45 (t, 1H), 7.35 (dd, 1H), 6.87 (d, 1H), 6.70 (d, 1H), 5.28 (s, 2H), 5.17 (s, 2H), 3.51 (s, 3H). 13C NMR (100 MHz, CDCl₃) 189.0, 160.7, 159.4, 148.7, 148.4, 137.4, 136.1, 131.3, 123.2, 115.5, 108.1, 106.1, 94.9, 66.6, 56.7.

### Preparation of Compound 7

To a 25 mL flask containing 1-isopropyl-1Hpyrazole-5-boronic acid (0.25 g) and 9 mL of dioxane was added compound 6 (0.5 g), water (2.75 mL), trans-dichloro bis(triphenylphosphine)palladium(II) (0.1225 g), and sodium bicarbonate (0.88 g). The mixture was heated under nitrogen at 82° C., and stirred until reaction was finished (extra amounts of 1-isopropyl-1Hpyrazole-5-boronic acid were added). The mixture was cooled and was added dioxane and water. Part of the solvent was removed on a rotavap at 40° C. under vacuum. The mixture was extracted with EtOAc and the organic layer was then washed with water. The combined filtrates were concentrated to afford a light brown oil of compound 7 (0.56 g, 90%).

1H NMR (400 MHz; CDCl₃) δ 10.57 (s, 1H), 8.67 (dd, 1H), 8.31 (d, 1H), 7.60 (s, 1H), 7.43 (dd, 1H), 7.37 (t, 1H), 6.81 (d, 1H), 6.48 (d, 1H), 6.35 (d, 1H), 5.26 (s, 2H), 5.04 (s, 2H), 4.60 (m, 1H), 3.50 (s, 3H), 1.46 (d, 6H). 13C NMR (100 MHz, CDCl₃) 189.0, 160.4, 159.7, 149.1, 148.2, 138.3, 138.1, 136.5, 135.9, 132.0, 123.7, 115.6, 108.1, 106.9, 106.2, 105.8, 94.9, 67.3, 56.7, 50.9, 22.9.

### Preparation of Voxelotor

To a solution of compound 7 (2.5 g) in THF (18.75 mL) was added conc. HCl (2.75 mL). The solution was stirred at rt until the reaction was complete. The mixture was added to a solution of NaHCO₃ (2.0 g) in water (170 mL), and the resulting precipitate was collected by filtration and dried to give crude solid Voxelotor (2.16 g, 98%).

1H NMR (400 MHz; CDCl₃) δ 11.92 (s, 1H), 10.36 (s, 1H), 8.73 (dd, 1H) 7.96 (dd, 1H), 7.58 (d, 1H), 7.40 (m, 1H), 6.55 (d, 1H), 6.33 (d, 1H), 6.25 (d, 1H), 5.07 (s, 2H), 4.65 (m, 1H), 1.46 (d, 6H). 13C NMR (100 MHz, CDCl₃) 193.8, 163.9, 160.9, 149.7, 149.2, 138.5, 138.4, 137.8, 136.9, 131.2, 123.5, 111.0, 110.9, 107.2, 102.0, 67.4, 50.9, 22.9.

1. A process for preparing Voxelotor 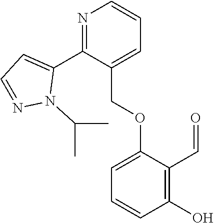 [SMILES: CC(C)n1nccc1-c1ncccc1COc1cccc(O)c1C=O]
or a salt or solvate thereof, comprising:
(a) reacting a compound of formula (I) 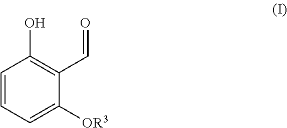
or a salt or solvate thereof, wherein R³ represents hydrogen or a hydroxyl protecting group, with a compound of formula (II) 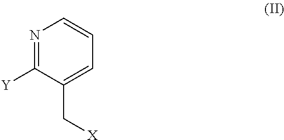
or a salt or solvate thereof, wherein
X is selected from OH, Cl, Br, I, OTf, OTs and OMs, and
Y is selected from Cl, Br, I, OTf and OMs;
to obtain a compound of formula (III) 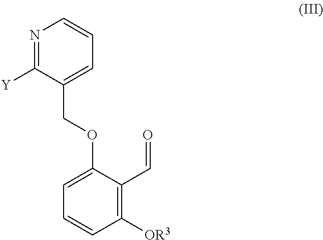
or a salt or solvate thereof;
(b) reacting a compound of formula (III), or a salt or solvate thereof, with a compound of formula (IV) 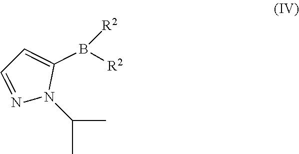
or a salt or solvate thereof, wherein each R² is independently selected from the group consisting of OH, C₁₋₆ alkyl, C₃₋₇ cycloalkyl, C₁₋₆ alkoxyl, or together they form a C₂₋₃ alkylenedioxy group optionally substituted by C₁₋₆ alkyl, or a benzyldioxy group optionally substituted by C₁₋₆ alkyl, or the —B(R²)₂ group is —BF₃K, to provide a compound of formula (V) 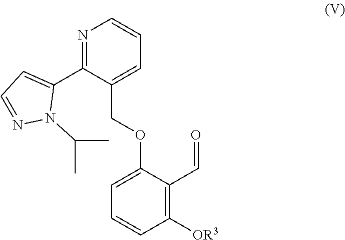
or a salt or solvate thereof; and
(c) if R³ in the compound of formula (V), or a salt or solvate thereof, is a hydroxyl protecting group, cleaving the hydroxyl protecting group to provide Voxelotor or a salt or solvate thereof.

2. Process according to claim 1, wherein X in the compound of formula (II), or a salt or solvate thereof, is selected from Cl, Br, I, OTf, OTs and OMs and step (a) is performed under alkylation reaction conditions.

3. Process according to claim 2, wherein step (a) is performed in the presence of a base and an organic polar solvent.

4. Process according to claim 1, wherein X in the compound of formula (II), or a salt or solvate thereof, is OH and step (a) is performed under Mitsunobu reaction conditions.

5. Process according to claim 4, wherein step (a) is performed in the presence of a first reagent selected from the group consisting of triphenylphosphine, tributylphosphine and trimethylphosphine, and a second reagent selected from the group consisting of group consisting of diisopropyl azodicarboxylate (DIAD), di-tert-butyl azodicarboxylate (DBAD), diethyl azodicarboxylate (DEAD), di-p-chlorobenzyl azodicarboxylate (DCAD), 1,1′-(azodicarbonyl)dipiperidine (ADDP), N,N,N′,N′-tetraisopropylazodicarboxamide (TIPA), N,N,N′,N′-tetramethylazodicarboxamide (TMAD) and 4,7-dimethyl-3,4,5,6,7,8-hexahydro-1,2,4,7-tetrazocin-3,8-dione (DHTD).

6. Process according to claim 1, wherein step (b) is performed in the presence of a base and a palladium catalyst.

7. Process according to claim 6, wherein the base is selected from alkaline and alkaline earth metal carbonates, bicarbonates, phosphates, acetates, alkoxides and hydroxides.

8. Process according to claim 6, wherein the palladium catalyst is selected from Pd(PPh₃)₄, Pd₂(dba)₃, Pd(OAc)₂, Pd(P^{t}Bu₃)₂, Pd(PCy₃)₂, Pd(PPh₃)₂Cl₂, Pd(P(o-tol)₃)₂Cl₂, Pd(PCy₃)₂Cl₂, Pd(P^{t}Bu₂Ph)₂Cl₂, Pd(P^{t}BuCy₂)₂Cl₂, Pd(P^{t}Bu₂ⁿBu₂)₂Cl₂, Pd(dppe)₂Cl₂, Pd(dppp)₂Cl₂, Pd(dppb)₂Cl₂, Pd(dppf)Cl₂, Pd(dtbpf)Cl₂, Pd(dcypp)Cl₂, [PdBr(P^{t}Bu₃)]₂, Pd(PhCN)₂Cl₂, Pd(CH₃CN)₂Cl₂, or a solvate thereof.

9. Process according to claim 1, wherein step (b) is performed in the presence of NaHCO₃ or Na₂CO₃, a catalyst selected from Pd(PPh₃)₂Cl₂, Pd(amphos)Cl₂, Pd(PCy₃)₂Cl₂ and Pd(PCy₃)₂, and a mixture of water and an organic solvent.

10. Process according to claim 1, wherein each R² in the compound of formula (IV) is independently selected from the group consisting of OH, C₁₋₆ alkoxyl, or together they form a C₂₋₃ alkylenedioxy group optionally substituted by C₁₋₆ alkyl or a benzyldioxy group optionally substituted by C₁₋₆ alkyl.

11. Process according to claim 1, wherein R³ in the compound of formula (I) is a group of formula —CH₂—O—R¹, wherein R¹ is a C₁₋₆ alkyl group.

12. Process according to claim 11, wherein the compound of formula (I), or a salt or solvate thereof, is obtained by a process comprising:
(a) reacting 1,3-benzenediol 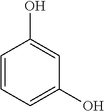
with a compound of formula R¹—O—CH₂-halide, wherein R¹ is a C₁₋₆ alkyl group, generated in situ by reacting a compound of formula R¹—O—CH₂—O—R¹ with a halide source; to obtain a compound of formula (VI) 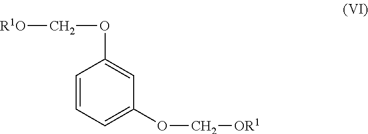
(b) formylating a compound of formula (VI), to obtain a compound of formula (VII) 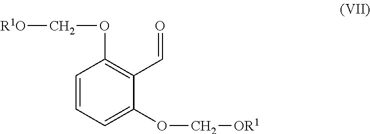
and
(c) cleaving one alkoxymethyl ether group in the compound of formula (VII), to obtain a compound of formula (I), or a salt or solvate thereof, wherein R³ is a group of formula —CH₂—O—R¹

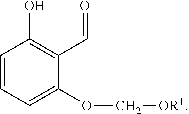

13. Process according to claim 1, wherein R³ is —CH₂—O—CH₃ (MOM).

14. Compound of formula (III′) 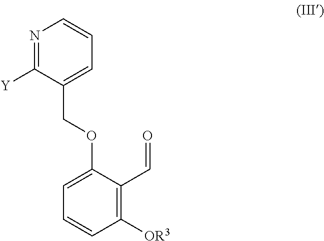
or a salt or solvate thereof, wherein
Y is selected from I, OTf and OMs, and
R³ represents hydrogen or a hydroxyl protecting group.

15. Compound according to claim 14, wherein R³ is selected from H and a group of formula:
—Si(R)(R′)(R″), wherein R, R′ and R″ are independently selected from C₁-C₆ alkyl, C₃-C₇ cycloalkyl, C₆-C₁₀ aryl, C₁-C₆ alkoxy and halogen;
—R, wherein R is selected from C₁-C₆ alkyl, C₆-C₁₀ aryl and (C₆-C₁₀)aryl(C₁-C₆)alkyl;
—CH₂—OR, wherein R is selected from C₁-C₆ alkyl, C₆-C₁₀ aryl and (C₆-C₁₀)aryl(C₁-C₆)alkyl;
—COR, wherein R is selected from C₁-C₆ alkyl, C₆-C₁₀ aryl and (C₆-C₁₀)aryl(C₁-C₆)alkyl; or
—COOR, wherein R is selected from C₁-C₆ alkyl, C₆-C₁₀ aryl and (C₆-C₁₀)aryl(C₁-C₆)alkyl.
